(19) United States
(12) Patent Application Publication
(10) Pub. No.: US 2019/0343092 A1
(43) Pub. Date: Nov. 14, 2019

(54) RODENT MODELS WITH AUTISTIC FEATURES

(71) Applicant: Tokyo Women's Medical University, Tokyo (JP)

(72) Inventors: Goichi MIYOSHI, Tokyo (JP); Mariko MIYATA, Tokyo (JP)

(21) Appl. No.: 16/409,052

(22) Filed: May 10, 2019

(30) Foreign Application Priority Data
May 11, 2018 (JP) 2018-092200

(51) Int. Cl. A01K 67/027; G01N 33/50

(52) U.S. Cl. CPC A01K 67/0276; G01N 33/5008; A01K 2227/105; A01K 2217/075; A01K 2267/03; A01K 2217/206

(57) ABSTRACT

The present invention (1) performs an operation of decreasing the number of copies of a FoxG1 gene from two copies to one copy in both differentiated excitatory neurons and differentiated inhibitory neurons present in a brain, or (2) performs, seven or more days after birth, an operation of increasing expression levels of a FoxG1 gene in both differentiated excitatory neurons and differentiated inhibitory neurons present in a brain to 1.2 to 2.0 times that of a wild type animal.

### TECHNICAL FIELD

The present invention relates to a rodent exhibiting an autistic characteristic and a method to prepare this animal model.

### BACKGROUND ART

Autism is a neurodevelopmental disorder which is drawing a social attention and is presently thought to occur in 1 in 70 children.

Autism is classified into syndromic types and idiopathic types based on clinical findings. Since the syndromic autism is caused by single-gene mutation, mutant model animals are developed by multiple research groups (Non-Patent Literature 1).

No model animals have been developed for idiopathic autism, which accounts for over 95% of autism cases, because of the lack of gene mutation commonly found in the disease.

Dysregulation of the FoxG1 gene has caught attention in recent years as a common pathological basis for syndromic and idiopathic autism. For example, a differentiation assay by using iPS cells, which are derived from a patient with idiopathic autism, shows an abnormality in FoxG1 gene expression levels in the process of neural differentiation. Importantly, these patient-derived cells do not carry obvious mutations in the FoxG1 gene itself, which suggests that FoxG1 dysregulation during development is a commonly shared path in the etiology of autism (Non-Patent Literature 2).

Note that the FoxG1 gene is located in the long arm of chromosome 14 and encodes a FoxG1 factor. The FoxG1 is an inhibitory transcription factor with a forkhead-shaped DNA binding domain and is known to play an important role in cerebral formation in the prenatal period.

Moreover, FoxG1 gene mutations have been observed in humans, and it has recently been shown that autism FoxG1 syndrome occurs in both cases of increase (locus duplication) and decrease (haploinsufficiency due to a point mutation) in the FoxG1 gene (Non-Patent Literatures 3 and 4).

### CITATION LIST

### Non-Patent Literature

[Non-Patent Literature 1] Nature Neuroscience, Vol. 19, No. 11, p. 1408-141.7 (2016)

[Non-Patent Literature 2] Cell, Vol. 162, Issue 2, p. 375-390 (2015)

[Non-Patent literature 3] Mol Syndromol. 2011 April; 2(3-5): 153-163

[Non-Patent literature 4] Eur J Hum Genet. 2011 January: 19(1): 102-7

### SUMMARY OF INVENTION

### Technical Problems

The development of therapeutics and early diagnosis inevitably requires model animals which reproduce the pathological basis commonly shared in human autism, but no such model animals have been developed. In fact, the International FoxG1 Foundation (URL:https://foxg1.org), established in 2012, has listed the development of model animals as one of its important aims.

### Solution to Problems

The present inventors have made earnest studies to develop such a model animal and found that mice with altered FoxG1 expression in specific cells of the brain in a specific time period show human autistic characteristics. The present invention has been made based on this finding.

Specifically, the present invention relates to the following [1] to [9].

[1]

A rodent exhibiting an autistic characteristic, wherein

the rodent is prepared by performing an operation of decreasing the number of copies of a FoxG1 gene from two copies to one copy in both differentiated excitatory neurons and differentiated inhibitory neurons present in a brain.

[2]

A method of preparing a rodent exhibiting an autistic characteristic, comprising the step of:

decreasing the number of copies of a FoxG1 gene from two copies to one copy in both differentiated excitatory neurons and differentiated inhibitory neurons present in a brain.

[3]

A rodent exhibiting an autistic characteristic, wherein

the rodent is prepared by performing, seven or more days after birth, an operation of increasing expression levels of a FoxG1 gene in both differentiated excitatory neurons and differentiated inhibitory neurons present in a brain to 1.2 to 2.0 times that of a wild type animal.

[4]

A method of preparing a rodent exhibiting an autistic characteristic, comprising the step of:

seven or more days after birth, increasing expression levels of a FoxG1 gene in both differentiated excitatory neurons and differentiated inhibitory neurons present in a brain to 1.2 to 2.0 times that of a wild type animal.

[5]

The rodent according to [1] or [3] described above, wherein the autistic characteristic is a social communication disorder.

[6]

The preparation method according to [2] or [4] described above, wherein the autistic characteristic is a social communication disorder.

[7]

The rodent according to [1] or [3] described above, wherein the rodent is a mouse.

[8]

The preparation method according to [2] or [4] described above, wherein the rodent is a mouse.

[9]

A method of screening a therapeutic drug or a prophylactic drug for autism, comprising the steps of:

administering a test substance to the rodent according to [1] or [3] described above or a rodent prepared by the method according to [2] or [4] described above; and selecting a therapeutic drug or a prophylactic drug based on an effect of the test substance on the autistic characteristic exhibited by the rodent.

### Advantageous Effects of Invention

As shown in Examples to be described later, the autistic model animal of present invention reproduces the pathological basis commonly found in human autism. Therefore, the present invention provides a research tool useful for drug screening, acquisition of knowledge necessary for developing therapeutics and early diagnosis for human autism, and the like.

### BRIEF DESCRIPTION OF THE DRAWINGS

FIG. 1 illustrates an overview of the mechanism of the decrease in the number of copies of the FoxG1 gene in both excitatory neurons and inhibitory neurons in the mice of Example 1 and the mechanism of confirming the decrease in the number of copies based on GFP.

FIG. 2 illustrates expression patterns of GFP in the brains of the mice of Example 1.

FIG. 3 illustrates an overview of 3-chamber sociability assay and the assay results for the mice of Examples 1 and 2.

FIG. 4 illustrates the results of staining the cerebral cortex barrel area of the mice of Example 2 with a FoxG1 antibody.

### DESCRIPTION OF EMBODIMENTS

Description is provided in further detail for the present invention which relates to a rodent exhibiting autistic characteristics (hereinafter also referred to as the “autistic model animal”).

The “autistic characteristics” refer to pathological conditions (symptoms) commonly appearing in human syndromic and idiopathic autism, and specifically refer to “social communication disorder” and “limited interest” classified as symptoms of autism spectrum disorder in the “Diagnostic and Statistical Manual of Mental Disorders, 5th Edition” (DSM-5), created by the American Psychiatric Association.

An autistic model animal can exhibit one or more of the “autistic characteristics” described above.

The determination of the presence or absence of the “autistic characteristics” can be made by the method used for model animals with syndromic autism. For example, the presence or absence of the “social communication disorder” can be determined by using the 3-chamber sociability assay (Nat Rev Neurosci. 2010 July; 11(7): 490-502). Description is provided for an overview of the 3-chamber sociability assay using mice.

(1) Preparing three chambers connected by passages, and providing the chambers at both ends with small cages 1 and 2 for placing the same sex and age mice (never-before-met mice) bred in cages different from that of the test target mouse.
(2) Allowing the test target mouse to freely move among the three chambers as an adaptation period (10 minutes).
(3) Subsequently placing the first never-before-met mouse in the small cage 1 to measure the degree of interest of the test target mouse in the first never-before-met mouse (10 minutes).
(4) Subsequently placing the second never-before-met mouse in the small cage 2 to measure which of the first never-before-met mouse (adapted in (3)) and the second never-before-met mouse the test target mouse is interested in (10 minutes).

In (3), the normal mouse is interested in the first never-before-met mouse and thus stays for a longer period of time in the chamber provided with the small cage 1. Meanwhile, in (4), the normal mouse is more interested in the second never-before-met mouse than in the first never-before-met mouse and thus stays for a longer period of time in the chamber provided with the small cage 2.

On the other hand, an autistic mouse shows behavior different from that of a normal mouse. Specifically, an autistic mouse avoids the first never-before-met mouse in (3) and thus stays for a longer period of time in the chambers than in the chamber provided with the small cage 1. In (4), there is no selectivity between the first never-before-met mouse and the second never-before-met mouse (newer mouse), and the autistic mouse stays for a longer period of time in the middle chamber without the never-before-met first and second mice. Therefore, the time spent in each chamber can be used as an index to determine the presence or absence of a social communication disorder.

In addition, the presence or absence of “limited interest” can be determined using the method described in the literature (Nat Rev Neurosci. 2010 July; 11(7): 490-502).

Examples of the “rodent” usable as an autistic model animal include mice, rats, naked mole rats, and guinea pigs. Mice are preferable among them because of their short breeding cycle, ability to breed all year round, and established handling techniques including genetic manipulation.

An autistic model animal can be prepared by an “operation of decreasing the number of copies of a FoxG1 gene in both differentiated excitatory neurons and differentiated inhibitory neurons present in a brain from two copies to one copy.”

The above operation can be carried out by a known conditional knock-out operation using the Cre/loxP system, for example the operation described in the literature (Dev Cell. 2004 January; 6(1): 7-28).

The Cre/loxP system is a known gene recombination system which uses a site-specific recombination reaction caused when Cre (DNA recombination enzyme) acts on the loxP sequence (DNA sequence)

The knock-out of the FoxG1 gene can be achieved by crossing a transgenic animal inserted with loxP sequences at both ends of the base sequence encoding protein information on the transcription factor FoxG1 and a transgenic animal expressing Cre under the control of a brain-specific promoter. In an individual obtained by the crossing, Cre expressed under the control of a brain-specific promoter in one genome acts on the loxP sequences to cause a deletion (knock-out) of the FoxG1 gene. Thus, the number of copies of the FoxG1 gene decreases from two copies to one copy,

In the present invention, knock-out is caused in one locus in the excitatory neurons and the inhibitory neurons in the “differentiated state” (conditional knock-out). The conditional knock-out can be achieved by crossing

(1) a transgenic animal A (female) carrying two copies of the FoxG1 gene inserted with loxP sequences at both ends and

(2) a transgenic animal B (male) in which the Nex locus of one genome has been inserted with a Cre gene and the other genome has been inserted with a fragment having a Cre gene under the control of the Dlx gene promoter.

In the transgenic animal B, the Nex gene promoter present in the Nex locus functions when undifferentiated proliferation cells differentiate into excitatory neurons, and the Dlx gene (homeobox gene) promoter functions when undifferentiated proliferation cells differentiate into inhibitory neurons.

All individuals obtained by the crossing carry the FoxG1 gene inserted with loxP sequences, and include the following four types classified based on the genetic composition derived from the transgenic animal B.

(i) carrying only the Cre gene under the control of the Nex gene promoter
(ii) carrying only the Cre gene under the control of the Dlx gene promoter
(iii) carrying both of the Cre genes of (i) and (ii)
(iv) carrying neither of the Cre genes of (i) and (ii)

The individuals (iii) are identified by genotyping using the genotyping method. In these individuals, the Nex gene promoter functions to express a downstream Cre gene when differentiated excitatory neurons occur in the brain, and the Dlx gene promoter functions to express a downstream Cre gene when differentiated inhibitory neurons occur in the brain. Thus, the number of copies of the FoxG1 gene decreases in both types of cells due to the Cre/loxP system.

Conditional knock-out animals can be selected by the method described in the literature (Neuron. 2012 Jun. 21; 74(6): 1045-58). An overview is explained as follows.

A gene of a different DNA recombinant enzyme Flpe is inserted in further downstream of the loxP sequence on the downstream side of the FoxG1 gene of each genome of the transgenic animal A. Each genome of the transgenic animal B is inserted with an expression stop cassette with both ends flanked by FRT sequences (DNA sequences) and is inserted in the downstream thereof with a green fluorescent protein (GFP) gene. Note that Flpe acts on the FRT sequences to cause a site-specific recombination reaction.

In the animals which experience conditional knock-out out of the animals obtained by the crossing of transgenic animals A and B, Flpe acts on the FRT sequences, which causes the deletion of the expression stop cassette to express the GFP gene, resulting in fluorescence. This mechanism can be used to select conditional knock-out animals with fluorescent light emission as an index and to identify the excitatory neurons and the inhibitory neurons for which the number of copies of the FoxG1 gene has decreased from two to one in the animal brain.

The base sequence of the FoxG1 gene is known, and for example, the base sequence of the mouse FoxG1 gene is registered on GenBank with an accession number of “NM_001160112” (URL: https://www.ncbi.nlm.nih.gov/nuccore/NM_001160112.1).

A loxP sequence can be inserted by a known gene targeting method. The loxP sequence is known, and it is possible to use, for example, the sequence corresponding to position 127 to position 160 of the sequence registered on GenBank with an accession number of “AF237862.1” (URL: https://www.ncbi.nlm.nih.gov/nuccore/AF237862.1).

The transgenic animal A can be prepared by the method described in the literature (Neuron. 2012 Jun. 21; 74(6): 1045-58).

The transgenic animal B can be obtained by crossing

(1) a transgenic animal (Nex-Cre animal) carrying one copy of genome inserted with a Cre gene in the downstream of the Nex gene promoter in the Nex locus and

(2) a transgenic animal (Dlx-Cre animal) carrying a genome inserted with a fragment having a Cre gene in the downstream of the Dlx gene promoter.

Nex-Cre animals can be prepared by the method described in the literature (Genesis. 2006 December; 44(12): 611-21) and are also available from Dr Klaus Nave at the Max Planck Institute for Experimental Medicine. Gottingen (Germany).

Dlx-Cre animals can be prepared by the method described in the literature (Neuron. 2006 August 17; 51(4): 455-66) or are commercially available (for example, mice of Jackson mouse Stock No: 008199|Dlx5/6-Cre (URL:https://wwwjax.org/strain/008199), The Jackson Laboratory).

In addition to the preparation methods described above, the autistic model animal can be prepared by “performing, seven or more days after birth, an operation of increasing expression levels of a FoxG1 gene in both differentiated excitatory neurons and differentiated inhibitory neurons present in a brain to 1.2 to 2.0 times that of a wild type animal.”

This operation can be achieved by crossing

(1) a transgenic animal C (female) carrying by homozygosity two copies each of the locus (R26-stop-tTA) which constitutively expresses transactivator tTA only when Cre recombination occurs and the locus (TRE-FoxG1) which carries the FoxG1 gene in the downstream of a promoter (TRE: Tet responsive element) that induces expression in a tTA-dependent manner and

(2) the transgenic animal B (male) described above.

The R26-stop-tTA of the transgenic animal C is inserted with an expression stop cassette flanked by loxP sequences. In the individuals obtained by the crossing, the undifferentiated proliferation cells differentiate into excitatory or inhibitory neurons to express Cre, which acts on the loxP sequences to delete the expression stop cassette, thereby inducing tTA. TRE-FoxG1 activated by this tTA induces the expression of the downstream FoxG1 gene.

Note that, in the present invention, the above-described operation of increasing the FoxG1 gene expression level is performed seven or more days after birth, and this can be achieved by providing the female with feed containing a tTA inhibitor (doxycycline) from the fetal age of zero days when a plug (index of successful mating) is attached to the female, and switching to normal feed on the seventh day after birth.

All individuals obtained by the crossing carry one copy each of the TRE-FoxG1 which expresses the FoxG1 gene in a tTA-dependent manner and the R26-stop-tTA which expresses tTA in a Cre-dependent mariner due to the loxP sequences, and include the following four types classified based on the genetic composition derived from the transgenic animal B.

(v) carrying only the Cre gene under the control of the Nex gene promoter
(vi) carrying only the Cre gene under the control of the Dlx gene promoter
(vii) carrying both of the Cre genes of (v) and (vi)
(viii) carrying neither of the Cre genes of (v) and (vi)

The individuals (vii) are identified by genotyping using the genotyping method. When these individuals are subjected to the induction operation of switching from feed containing a tTA inhibitor (doxycycline) to normal feed seven or more days after birth, tTA which has been inactivated by the tTA inhibitor is activated, resulting in an increase in the expression level of the FoxG1 gene.

The increase in the expression level of the FoxG1 gene in the autistic model animal of the present invention is 1.2 to 2.0 times and preferably 1.5 to 2.0 times the expression level of the FoxG1 gene in of a wild type animal. Note that although a 1.2 to 2.0 fold increase in the expression level can be achieved by the operation using the transgenic animals B and C, the degree of increase in the expression level of the FoxG1 gene may be adjusted by adjusting the amount of the tTA inhibitor blended in the feed and increasing the number of copies of the TRE-FoxG1 in the transgenic animal C. Additionally, it is possible to quantify the increase in the expression level of the FoxG1 gene by immunostaining using a FoxG1 antibody.

The sequences involved in the preparation of the transgenic animal C having the R26-stop-tTA are known, and

usable examples of R26 include the sequence of the R26 locus (Gt(ROSA)26Sor) present in mouse chromosome 6 (sequence registered on GenBank with an accession number of “MGI:MGI:104735” (URL: hitp://www.informatics.jax.org/marker/MGI: 104735)),

usable examples of the expression stop cassette (stop) include the sequence registered on GenBank with an accession number of “KX803821.1” (URL:https://www.ncbi.mlm.nih.gov/nuccore/KX803821.1), and

usable examples of tTA include the sequence registered on GenBank with an accession nwnber of “KX766191” (URL:https://www.ncbi.nlm.nih.gov/nuccore/KX766191. 1).

The sequence of TRE is also known, and usable examples thereof include the sequence registered on GenBank with an accession number of “MG874803” (URL: https://www.ncbi.nlm.nih.gov/nuccore/MG874803.1).

The transgenic animal C is obtained by crossing

(1) a transgenic animal (R26-stop-tTA animal) carrying one copy of genome inserted with, in the R26 locus, an expression stop cassette flanked by loxP sequences in the downstream of an R26 gene promoter and with transactivator tTA in further downstream thereof and

(2) a transgenic animal (TRE-FoxG1 animal) carrying a genome inserted with a fragment having the FoxG1 gene in the downstream of a promoter (TRE) that induces expression in a tTA-dependent manner.

The R26-stop-tTA animal can be prepared by the method described in the literature Neurobiol Dis 29(3): 400-8) or is commercially available (for example, mice of Jackson mouse Stock No: 008600 Gt(ROSA)26Sortml(tTA)Roos (URL: https://www.jax.org/strain/008600), The Jackson Laboratory).

The TRE-FoxG1 animal can be prepared by the method described in the literature (J Neurosci. 2002 Aug. 1; 22(15): 6526-36).

The autistic model animal of the present invention can be used as a research tool in the development of therapeutics and early diagnosis for human autism.

For example, it is possible to carry out screening of a therapeutic drug or a prophylactic drug for human autism by administering a test substance to the autistic model animal of the present invention and utilizing as the basis the effects of the test substance on the autistic characteristics exhibited by the model animal before administration.

Next, specific description is provided for the effects of the present invention with reference to Examples, but the present invention is not limited to Examples,

### EXAMPLES

### Example 1

Example 1 applied a known conditional knock-out operation (Dev Cell. 2004 January; 6(1): 7-28) to a mouse to prepare an autistic mouse subjected to the “operation of decreasing the number of copies of a FoxG1 gene in both differentiated excitatory neurons and differentiated inhibitory neurons present in a brain from two copies to one copy.”

### (1) Transgenic Animal A

The transgenic animal A (female) was prepared from a mouse (trade name: floxed-FoxG1, source: New York university) in accordance with the method described in the literature (Neuron. 2012 Jun. 21; 74(6): 1045-58).

The transgenic animal A carried two copies of the FoxG1 gene inserted with loxP sequences at both ends, and was further inserted with the Flpe gene in further downstream of the loxP sequence on the downstream side of the FoxG1 gene of each copy.

The sequence of the FoxG1 gene was the sequence registered on GenBank with an accession number of “NM_001160112.”

The loxP sequence was the sequence corresponding to position 127 to position 160 of the sequence registered on GenBank with an accession number of “AF237862.1.”

The sequence of the Flpe gene was the sequence registered on GenBank with an accession number of “GU253314.”

### (2) Transgenic Animal B

The transgenic animal B was prepared by crossing a Nex-Cre animal (transgenic animal carrying one copy of genome inserted with a Cre gene in the downstream of the Nex gene promoter in the Nex locus) and a Dlx-Cre animal (transgenic animal carrying a genome inserted with a fragment having a Cre gene in the downstream of the Dlx gene promoter).

The Nex-Cre animal used was a mouse prepared in accordance with the method described in the literature (Genesis. 2006 December; 44(12): 611-21).

The Dlx-Cre animal used was a mouse of Jackson mouse Stock No: 008199 Dlx5/6-Cre (URL: https://www.jax,org/strain/008199), The Jackson Laboratory.

The transgenic animal B was selected by the genotyping method from 40 male mice obtained by the crossing. In the transgenic animal B, the Nex locus of one genome had been inserted with a Cre gene and the other genome had been inserted with a fragment having a Cre gene under the control of the Dlx gene promoter.

The Cre gene sequence in each of the Nex-Cre animal and the Dix-Cre animal was the sequence registered on GenBank with an accession number of “X03453” (URL: https://www.ncbi.gov/nuccore/NC_005856. 1).

In the Nex-Cre animal, the sequence of the Nex locus present in mouse chromosome 6 before the insertion of Cre was the sequence registered on GenBank with an accession number of “NM_009717”(http://asia.ensembl.org/Mus_musculus/Gene/Summary?db=core;g=ENSMUSG000000 37984;r=6:55677822-55681263;t=ENSMUST00000044767).

The Dlx gene promoter sequence in the Dlx-Cre animal was the sequence registered on GenBank with an accession number of “AF201695” (https://www.ncbi.nlm.nih.gov/nuccore/AF201695.4).

(3) Operation of Decreasing Number of Copies of FoxG1 Gene from Two Copies to One Copy

Fifteen transgenic animals A (female) and six transgenic animals B (male) were crossed. Selected by the genotyping method from the obtained 125 mice were individuals (34 mice) carrying both (i) the Cre gene under the control of the Nex gene promoter and (ii) the Cre gene under the control of the Dix gene promoter.

It was observed based on fluorescent light emission that the number of copies of the FoxG1 gene had decreased from two copies to one copy in both excitatory neurons and inhibitory neurons of the selected mice.

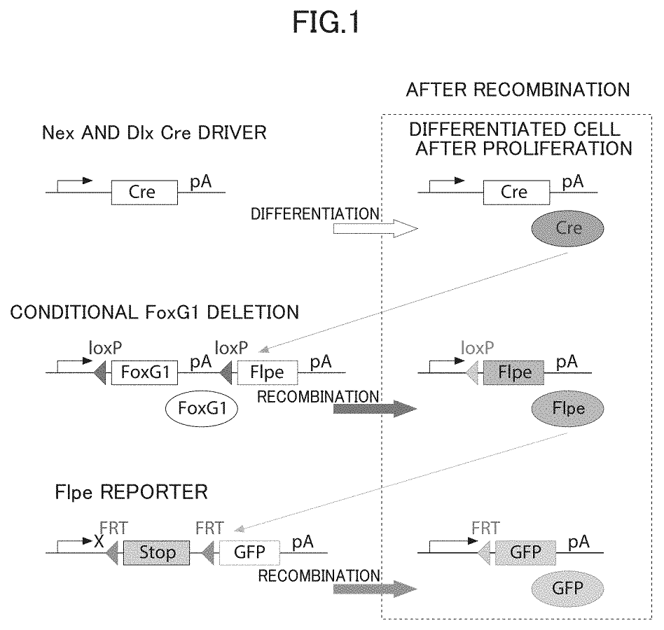

Here, description is provided for the mechanism of fluorescent light emission based on FIG. 1. The DNA recombinant enzyme Cre is not expressed in undifferentiated proliferating cells. However, when the proliferating cells differentiate into excitatory neurons, the Nex gene promoter becomes active to express the downstream Cre gene, and when the proliferating cells differentiate into inhibitory neurons, the Dlx gene promoter expresses the downstream Cre gene (upper part of the figure). The expressed Cre acts on the loxP sequences to cause recombination (deletion) of the FoxG1 gene, which decreases the number of copies of the FoxG1 gene from two to one (left of the middle part of the figure). Moreover, when the recombination (deletion) of the FoxG1 gene takes place correctly, the promoter of the FoxG1 locus originally regulating the expression of the FoxG1 gene in tum expresses the downstream DNA recombinant enzyme Flpe (right of the middle part of the figure). Since Flpe deletes the stop cassette flanked by FRT sequences (left of the lower part of the figure), the downstream green fluorescent protein (GFP) gene becomes expressed to produce fluorescence (right of the lower part of the figure)

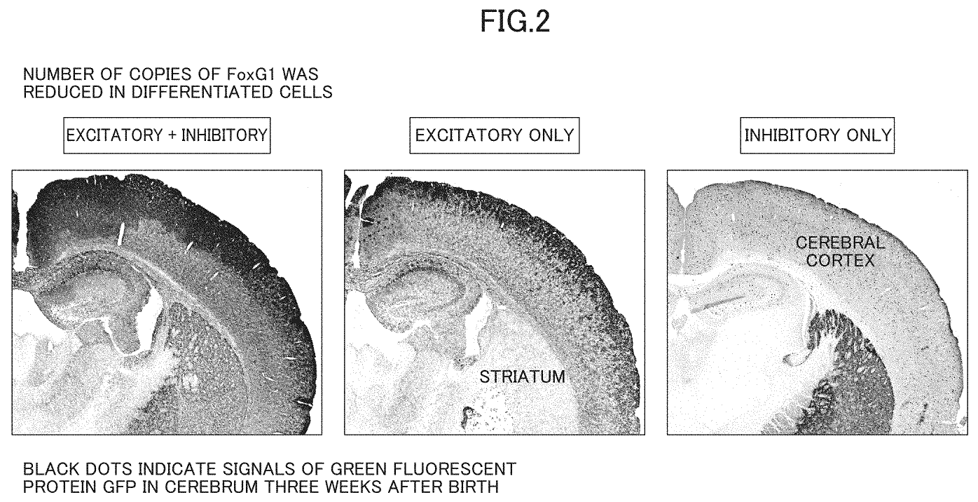

FIG. 2 illustrates the expression pattern of GFP in the brain of each of the selected mouse three weeks after birth (left), the mouse whose number of copies of the FoxG1 gene had been decreased only in excitatory neurons (middle), and the mouse whose number of copies of the FoxG1 gene had been decreased only in inhibitory neurons (right). A GFP-recognizing antibody (trade name: Rat GFP Antibody #GF090R, supplier name: Nacalai Tesque Inc.) was used for staining to measure the GFP expression present in excitatory neurons and in inhibitory neurons. The GFP signals are shown black.

About 80 percent of the cerebral cortex was occupied by excitatory neurons (the remainder are inhibitory neurons). While the striatum was composed only of inhibitory neurons, the GFP signals were observed only in the cerebral cortex for the mouse whose number of copies of the FoxG1 gene had been decreased only in excitatory neurons (middle of FIG. 2).

The GFP signals were observed only in the striatum and part of the cerebral cortex for the mouse whose number of copies of the FoxG1 gene had been decreased only in inhibitory neurons (right of FIG. 2).

On the other hand, the staining results of the selected mouse (left of FIG. 2) were as if the combination of the staining results described above (specifically, the GFP signals were observed in the cerebral cortex and the striatum). It is understood from this figure that, in the selected mouse, the number of copies of the FoxG1 gene decreased in both excitatory and inhibitory neurons.

### (4) Evaluation of Autistic Characteristics

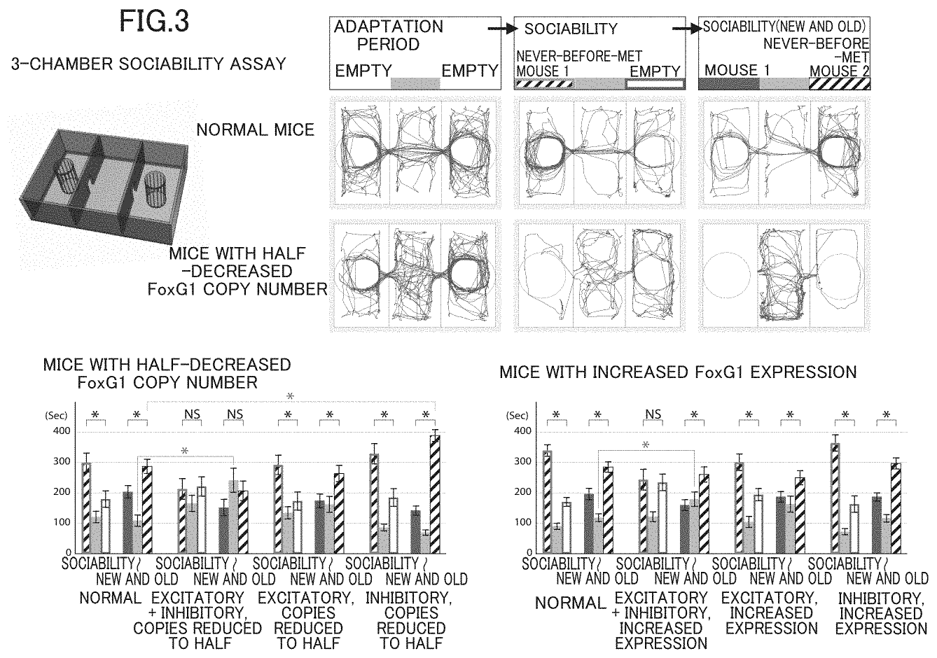

The mice obtained in (3) were evaluated by 3-chamber sociability assay (Nat Rev Neurosci. 2010 July; 11(7): 490-502) for the presence or absence of a social communication disorder, which is an autistic characteristic. Description is provided for the specific procedures based on FIG. 3.

(i) Preparing three chambers connected by passages, and providing the chambers at both ends with small cages 1 and 2 for placing the same sex and age mice (never-before-met mice) bred in cages different from that of the test target mouse (mouse obtained in (3)) (upper left of FIG. 3).
(ii) Allowing the test target mouse to freely explore the three chambers as an adaptation period (10 minutes) (“Adaptation Period” in the upper right of FIG. 3).
(iii) Subsequently placing the first never-before-met mouse in the small cage 1 to measure the degree of interest of the test target mouse in the first never-before-met mouse (10 minutes) (“Sociability” in the upper right of FIG. 3).
(iv) Subsequently placing the second never-before-met mouse in the small cage 2 to measure which of the first never-before-met mouse (adapted in (iii)) and the second never-before-met mouse the test target mouse was interested in (10 minutes) (“Sociability (New and Old)” in the upper right of FIG. 3).

In (iii), the normal mouse was interested in the first never-before-met mouse and staved for a longer period of time in the chamber provided with the small cage 1 (bar graphs in the lower left of FIG. 3, “Sociability” of “Normal”). Meanwhile, in (iv), the normal mouse was more interested in the second never-before-met mouse than in the first never-before-met mouse and stayed for a longer period of time in the chamber provided with the small cage 2 (bar graphs in the lower left of FIG. 3, “New and Old” of “Normal”).

On the other hand, the test target mouse showed behavior different from that of a normal mouse. Specifically, the test target mouse avoided the first never-before-met mouse in (iii) and stayed for a longer period of time in the chambers than in the chamber provided with the small cage 1 (bar graphs in the lower left of FIG. 3, “Sociability” of “Excitatory+Inhibitory, Copies Reduced to Half”). In addition, in (iv), there was no selectivity between the first never-before-met mouse and the second never-before-met mouse (newer mouse), and the test target mouse stayed for a longer period of time in the middle chamber without the never-before-met first and second mice (bar graphs in the lower left of FIG. 3, “New and Old” of “Excitatory+inhibitory, Copies Reduced to Half”).

It was determined based on those behaviors different from normal mice that the test target mouse (mouse obtained in (3)) exhibited a social communication disorder.

Note that sociability was approximately the same as that of normal mice for the “mouse whose number of copies of the FoxG1 gene was decreased from two copies to 1 copy only in differentiated excitatory neurons” (bar graphs in the lower left of FIG. 3, “Excitatory, Copies Reduced to Half”) and the “mouse whose number of copies of the FoxG1 gene was decreased from two copies to 1 copy only in differentiated inhibitory neurons” (bar graphs in the lower left of FIG. 3, “Inhibitory, Copies Reduced to Half”). Note that the symbol “*” in the bar graphs in the lower left of FIG. 3 means P<0.05.

### Example 2

Example 2 prepared an autistic mouse by “performing, seven or more days after birth, an operation of increasing expression levels of a FoxG1 gene in both differentiated excitatory neurons and differentiated inhibitory neurons present in a brain to 1.2 to 2.0 times that of a wild type animal.”

### (1) Transgenic Animal C

The R26-stop-tTA animal used was a commercially available mouse (Jackson mouse Stock No: 008600 Gt(ROSA)26Sortml(tTA)Roos CURL: https://www.jax.org/strain/008600), The Jackson Laboratory).

The TRE-FoxG1 animal (mouse) was prepared in accordance with the method described in the literature (J Neurosci. 2002 Aug. 1; 22(15): 6526-36).

The R26-stop-tTA mouse and the TRE-FoxG1 mouse were crossed to obtain eight mice, and the eight mice were further crossed to select by the genotyping method the transgenic animal C carrying by homozygosity two copies each of R26-stop-tTA and TRE-FoxG1.

The sequence of the R26 locus was the sequence registered on GenBank with an accession number of “MGI:MGI:104735.”

The sequence of the expression stop cassette (stop) was the sequence registered on GenBank with an accession number of “10(81)3821.1.”

The sequence of CIA was the sequence registered on GenBank with an accession number of “KX766191.”

The sequence of TRE was the sequence registered on GenBank with an accession number of “MG874803.”

### (2) Transgenic Animal B

The transgenic mice prepared by the procedures described in Example 1 were used.

### (3) Operation of Increasing Expression Level of FoxG1 Gene

Nine transgenic animals C (female) and three transgenic animals B (male) were crossed. Selected by the genotyping method from the obtained 78 mice were individuals (17 mice) carrying both (i) the Cre gene under the control of the Nex gene promoter and (ii) the Cre gene under the control of the Dlx gene promoter.

The selected individuals were provided with feed added with a tTA inhibitor (doxycycline) (trade name: Mod LabDiet 5001 w/200 PPM Doxycycline, supplier: TestDiet) from the fetal age of zero days, when plugs (index of successful mating) were attached to the parent transgenic animals C (female), until sixth day after birth. Only normal feed (not containing the tTA inhibitor) was provided seven or more days after birth. These feeding procedures were used to perform the operation of increasing the expression level of the FoxG1 gene seven or more days after birth.

On the fourteenth day after birth, measurement was carried out in accordance with the following procedures on the expression levels of a FoxG1 gene in both differentiated excitatory neurons and differentiated inhibitory neurons present in a brain of each of the selected mice, and the expression levels were compared with those of a wild type mouse. The measurement target selected was the barrel area whose site in the cerebral cortex is easily identified and which has both excitatory neurons and inhibitory neurons.

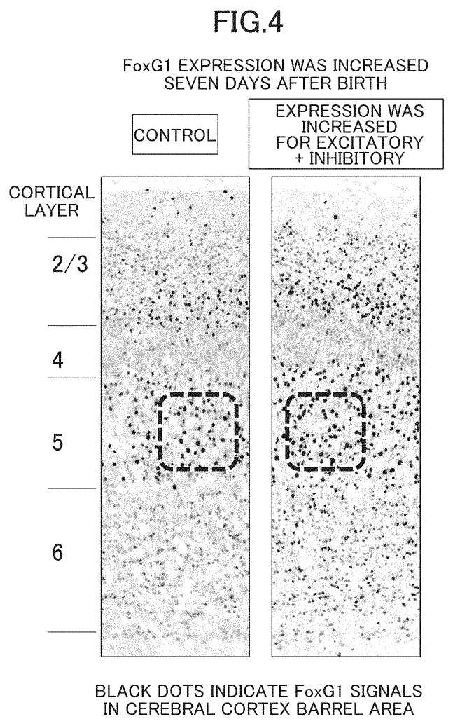

Cerebral sections including the barrel area were prepared and stained with a FoxG1 antibody (trade name: Rabbit FoxG1 Antibody, supplier name: Neuracell). FIG. 4 illustrates the staining results. The dotted line enclosures in FIG. 4 are each the fifth layer of the barrel area in the measurement target area.

The pixels showing the FoxG1 gene expression were defined as ones having a signal intensity of 157 or higher in the 256 steps (ones having intensities of top 100) out of the dot-shaped stains (pixels) observed in the fifth layer in the barrel area.

In the control mouse (wild type mouse), the number of pixels showing the FoxG1 gene expression out of all 46464 pixels in the barrel area was 2736 (0.05888 relative to the total number of pixels).

On the other hand, in the mouse subjected to the operation of increasing the expression level of the FoxG1 gene, the number of pixels showing the FoxG1 gene expression out of all 52528 pixels in the barrel area was 5332 (0.1015077 relative to the total number of pixels).

Therefore, in the mouse subjected to the operation of increasing the expression level of the FoxG1 gene illustrated in FIG. 4, the expression level of the FoxG1 gene had increased to 1.723 times (0.1015077/0.05888) that of a wild type mouse.

Note that a similar increase in expression level was also observed in cortical layers other than the fifth layer of the barrel area, as well as in other visual and motor areas.

### (4) Evaluation of Autistic Characteristics

The mouse observed in (3) to have an increased expression level of the FoxG1 gene (test target mouse) was evaluated by 3-chamber sociability assay described in Example 1 for the presence or absence of a social communication disorder.

In (iii), the normal mouse was interested in the first never-before-met mouse and stayed for a longer period of time in the chamber provided with the small cage 1 (bar graphs in the lower right of FIG. 3, “Sociability” of “Normal”). Meanwhile, in (iv), the normal mouse was more interested in the second never-before-met mouse than in the first never-before-met mouse and stayed for a longer period of time in the chamber provided with the small cage 2 (bar graphs in the lower right of FIG. 3, “New and Old” of “Normal”).

On the other hand, the test target mouse showed behavior different from that of a normal mouse, Specifically, the test target mouse avoided the first never-before-met mouse in (iii) and stayed for a longer period of time in the chambers than in the chamber provided with the small cage I (bar graphs in the lower right of FIG. 3, “Sociability” of “Excitatory+Inhibitory, Increased Expression”), In addition, in (iv), although there was a tendency of showing affinity to the second never-before-met mouse (newer mouse) than to the first never-before-met mouse, the test target mouse stayed for a longer period of time in the middle chamber without the never-before-met first and second mice (bar graphs in the lower right of FIG. 3, “New and Old” of “Excitatory+Inhibitory, Increased Expression”).

It was determined based on those behaviors different from normal mice that the test target mouse exhibited a social communication disorder.

Note that sociability was approximately the same as that of normal mice for the “mouse whose expression level of the FoxG1 gene was increased (1.2 to 2.0 times) only in differentiated excitatory neurons following the seventh day after birth” (bar graphs in the lower right of FIG. 3, “Excitatory, Increased Expression”) and the “mouse whose expression level of the FoxG1 gene was increased (1.2 to 2.0 times) only in differentiated inhibitory neurons following the seventh day after birth” (bar graphs in the lower right of FIG. 3, “Inhibitory, Increased Expression”). Note that the symbol “*” in the bar graphs in the lower right of FIG. 3 means P<0.05.

### INDUSTRIAL APPLICABILITY

The present invention can be used as a tool in the development of e.g. therapeutics and early diagnosis for human autism.

1. A rodent exhibiting an autistic characteristic, wherein
the rodent is prepared by performing an operation of decreasing the number of copies of a FoxG1 gene from two copies to one copy in both differentiated excitatory neurons and differentiated inhibitory neurons present in a brain.

2. A method of preparing a rodent exhibiting an autistic characteristic, comprising the step of:
decreasing the number of copies of a FoxG1 gene from two copies to one copy in both differentiated excitatory neurons and differentiated inhibitory neurons present in a brain.

3. A rodent exhibiting an autistic characteristic, wherein
the rodent is prepared by performing, seven or more days after birth, an operation of increasing expression levels of a FoxG1 gene in both differentiated excitatory neurons and differentiated inhibitory neurons present in a brain to 1.2 to 2.0 times that of a wild type animal.

4. A method of preparing a rodent exhibiting an autistic characteristic, comprising the step of:
seven or more days after birth, increasing expression levels of a FoxG1 gene in both differentiated excitatory neurons and differentiated inhibitory neurons present in a brain to 1.2 to 2.0 times that of a wild type animal.

5. The rodent according to claim 1, wherein
the autistic characteristic is a social communication disorder.

6. The preparation method according to claim 2, wherein
the autistic characteristic is a social communication disorder.

7. The rodent according to claim 1, wherein
the rodent is a mouse.

8. The preparation method according to claim 2, wherein
the rodent is a mouse.

9. A method of screening a therapeutic drug or a prophylactic drug for autism, comprising the steps of:
administering a test substance to the rodent according to claim 1; and
selecting a therapeutic drug or a prophylactic drug based on an effect of the test substance on the autistic characteristic exhibited by the rodent.

10. The rodent according to claim 3, wherein
the autistic characteristic is a social communication disorder.

11. The preparation method according to claim 4, wherein
the autistic characteristic is a social communication disorder.

12. The rodent according to claim 3, wherein
the rodent is a mouse.

13. The preparation method according to claim 4, wherein
the rodent is a mouse.

14. A method of screening a therapeutic drug or a prophylactic drug for autism, comprising the steps of:
administering a test substance to the rodent according to claim 3; and
selecting a therapeutic drug or a prophylactic drug based on an effect of the test substance on the autistic characteristic exhibited by the rodent.

15. A method of screening a therapeutic drug or a prophylactic drug for autism, comprising the steps of:
administering a test substance to a rodent prepared by the method according to claim 2; and
selecting a therapeutic drug or a prophylactic drug based on an effect of the test substance on the autistic characteristic exhibited by the rodent.

16. A method of screening a therapeutic drug or a prophylactic drug for autism, comprising the steps of:
administering a test substance to a rodent prepared by the method according to claim 4; and
selecting a therapeutic drug or a prophylactic drug based on an effect of the test substance on the autistic characteristic exhibited by the rodent.
